(19) United States
(12) Patent Application Publication
(10) Pub. No.: US 2023/0355769 A1
(43) Pub. Date: Nov. 9, 2023

(54) COMPOSITIONS, CONTAINERS, AND METHODS RELATED TO FLAVYLIUM EXCIPIENTS

(71) Applicant: Natural Extraction Systems, LLC, Boulder, CO (US)

(72) Inventor: C. Russell Thomas, Boulder, CO (US)

(73) Assignee: Natural Extraction Systems, LLC, Boulder, CO (US)

(21) Appl. No.: 18/306,223

(22) Filed: Apr. 24, 2023

Related U.S. Application Data
(60) Provisional application No. 63/333,962, filed on Apr. 22, 2022.

(51) Int. Cl. A61K 47/22; A61K 31/00

(52) U.S. Cl. CPC A61K 47/22; A61K 31/658

(57) ABSTRACT

Various aspects of this disclosure relate to a composition, comprising a liquid phase that consists of chemical species that comprise tetrahydrocannabinol, one or more flavyliums, and a solvent, wherein each chemical species of the liquid phase has a concentration in the liquid phase; the tetrahydrocannabinol is dissolved in the liquid phase at a concentration of at least 0.1 percent and no greater than 8 percent by mass; each flavylium of the one or more flavyliums is a substituted 2-phenylchromenylium that consists of carbon, hydrogen, and oxygen atoms; the solvent is miscible with water; and the liquid phase comprises the solvent at a greater concentration by mass than any other chemical species of the liquid phase.

### CROSS-REFERENCE TO RELATED APPLICATIONS

This patent application claims priority to U.S. Provisional Patent Application No. 63/333,962, filed Apr. 22, 2022, which is incorporated by reference in its entirety.

### BACKGROUND

The oral administration of tetrahydrocannabinol displays poor bioavailability and pharmacokinetics relative to inhalational administration. Formulations that improve the bioavailability and pharmacokinetics of tetrahydrocannabinol for oral administration are desirable.

### SUMMARY

Various aspects of this disclosure relate to the finding that tetrahydrocannabinol formulations comprising a water-miscible solvent and a flavylium excipient dramatically improve the bioavailability and pharmacokinetics of the tetrahydrocannabinol following oral administration.

Such formulations comprise the tetrahydrocannabinol and the flavylium excipient(s) dissolved in the water-miscible solvent. Following oral consumption, extracellular water from saliva rapidly dilutes the water-miscible solvent. Tetrahydrocannabinol is insoluble in water, and the dilution of the water-miscible solvent with extracellular water causes the tetrahydrocannabinol to partition out of a formulation following oral consumption. Flavylium excipients can then favor the partitioning of the tetrahydrocannabinol into the epithelial lining of the gastrointestinal tract, for example, by non-covalent interactions including pi-stacking and hydrogen-bonding interactions. A flavylium excipient can favor, for example, (i) interactions between tetrahydrocannabinol and the flavylium excipient that compete with interactions between the tetrahydrocannabinol and other molecules of the gastrointestinal tract, and (ii) interactions between the epithelial lining of the gastrointestinal tract and the flavylium excipient that compete with interactions between the excipient and other molecules of the gastrointestinal tract, which combine to favor the partitioning of the tetrahydrocannabinol into the epithelial lining of the gastrointestinal tract.

The preceding paragraph provides a mechanism that accounts for the improved bioavailability and pharmacokinetics obtainable using the compositions and methods of this disclosure, but the mechanism does not limit this disclosure or any patent claim that matures from this document. The flavylium excipients of this disclosure carry positive charges that may favor interactions with the negatively-charged epithelial lining of the gastrointestinal tract, which may contribute to the improved properties of the compositions of this disclosure through mechanisms that are consistent with those described in the preceding paragraph.

### DETAILED DESCRIPTION

Various aspects of this disclosure relate to a composition, comprising a liquid phase that consists of chemical species that comprise tetrahydrocannabinol, one or more flavyliums, and a solvent, wherein each chemical species of the liquid phase has a concentration in the liquid phase; the tetrahydrocannabinol is dissolved in the liquid phase at a concentration of at least 0.1 percent and no greater than 8 percent by mass; each flavylium of the one or more flavyliums is a substituted 2-phenylchromenylium that consists of carbon, hydrogen, and oxygen atoms; the solvent is miscible with water; and the liquid phase comprises the solvent at a greater concentration by mass than any other chemical species of the liquid phase.

“Comprising” and “comprise” refer to open sets, for example, such that a composition that comprises a liquid phase can also comprise a solid phase.

“Consist(s)” refers to close sets, for example, such that a substituted 2-phenylchromenylium that consists of carbon, hydrogen, and oxygen atoms cannot also comprise a nitrogen atom.

In some embodiments, each flavylium of the one or more flavyliums is a substituted 2-phenylchromenylium, in which 0, 1, or 2 hydrogen atoms of a 2-phenylchromenylium cation are independently substituted with a sugar such that the flavylium is an aglycoside, 3-O-glycoside, or 3,5-O-diglycoside, respectively; each sugar is selected from 6-O-acetylglucose, 6-O-(para-caffeoyl)glucose, 6-O-(para-coumaroyl)glucose, arabinose, galactose, glucose, rhamnose, and rutinose; and other hydrogen atoms of the 2-phenylchromenylium cation are optionally and independently substituted with hydroxy or methoxy. In some specific embodiments, each flavylium of the one or more flavyliums is a substituted 2-phenylchromenylium selected from 5-desoxymalvidin, 5-desoxypeonidin, 6-hydroxycyanidin, antirrhinin, apigeninidin, aurantinidin, callistephin, capensinidin, chrysanthemin, columnidin, cyanidin, cyanidin 3-O-(6-acetyl)glucoside, cyanidin 3-O-(6-p-coumaroyl)glucoside, cyanin, delphin, delphinidin, delphinidin 3-O-(6-acetyl)glucoside, delphinidin 3-O-(6-p-coumaroyl)glucoside, delphinidin 3-O-rhamnoside, diosmetinidin, europinidin, fisetinidin, gesneridin, guibourtinidin, hirsutidin, ideain, kaempferidinidin, luteolinidin, malvidin, malvidin 3-O-(6-acetyl)glucoside, malvidin 3-O-(6-p-caffeoyl)glucoside, malvidin 3-O-(6-p-coumaroyl)glucoside, malvin, myrtillin, oenin, pelargonidin, pelargonin, peonidin, peonidin 3-O-(6-acetyl)glucoside, peonidin 3-O-(6-p-caffeoyl)glucoside, peonidin 3-O-(6-p-coumaroyl)glucoside, peonidin 3-O-glucoside, peonin, petunidin, petunidin 3-O-(6-acetyl)galactoside, petunidin 3-O-(6-acetyl)glucoside, petunidin 3-O-(6-p-coumaroyl)glucoside, petunidin 3-O-arabinoside, petunidin 3-O-galactoside, petunidin 3-O-glucoside, petunidin 3-O-rhamnoside, petunidin 3-O-rutinoside, petunin, primulin, pulchellidin, pulchellidin 3-O-glucoside, pulchellidin 3-rhamnoside, robinetinidin, rosinidin, tricetinidin, and tulipanin. 2-phenylchromenylium cation is depicted in Structure I below. It contains a full, delocalized positive charge of +1, which is historically depicted as residing on its oxygen atom. 2-phenylchromenylium cation has the chemical formula C₅H₁₁⁺ and a molecular weight of 207 atomic mass units.

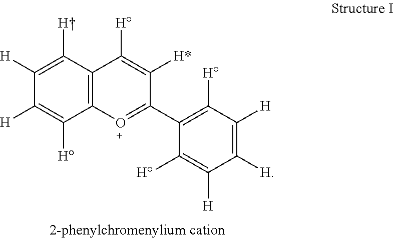

* denotes a hydrogen that can optionally be substituted with a sugar to result in a 3-O-glycoside

† denotes a hydrogen that can optionally be substituted with a sugar to result in a 5-O-glycoside

° denotes four hydrogens that preferably remain unsubstituted in the flavyliums of this disclosure

In some embodiments, the one or more flavyliums comprise one or more substituted 2-phenylchromenyliums, in which 0, 1, or 2 hydrogen atoms of a 2-phenylchromenylium cation are independently substituted with a sugar such that the flavylium is an aglycoside, 3-O-glycoside, or 3,5-O-diglycoside, respectively; each sugar is selected from arabinose, galactose, glucose, rhamnose, and rutinose; and other hydrogen atoms of the 2-phenylchromenylium cation are optionally and independently substituted with hydroxy or methoxy. In some specific embodiments, the one or more flavyliums comprise one or more substituted 2-phenylchromenyliums selected from 5-desoxymalvidin, 5-desoxypeonidin, 6-hydroxycyanidin, antirrhinin, apigeninidin, aurantinidin, callistephin, capensinidin, chrysanthemin, columnidin, cyanidin, cyanin, delphin, delphinidin, delphinidin 3-O-rhamnoside, diosmetinidin, europinidin, fisetinidin, gesneridin, guibourtinidin, hirsutidin, ideain, kaempferidinidin, luteolinidin, malvidin, malvin, myrtillin, oenin, pelargonidin, pelargonin, peonidin, peonidin 3-O-glucoside, peonin, petunidin, petunidin 3-O-arabinoside, petunidin 3-O-galactoside, petunidin 3-O-glucoside, petunidin 3-O-rhamnoside, petunidin 3-O-rutinoside, petunin, primulin, pulchellidin, pulchellidin 3-O-glucoside, pulchellidin 3-rhamnoside, robinetinidin, rosinidin, tricetinidin, and tulipanin.

In some embodiments, each flavylium of the one or more flavyliums is a substituted 2-phenylchromenylium, in which 0, 1, or 2 hydrogen atoms of a 2-phenylchromenylium cation are independently substituted with a sugar such that the flavylium is an aglycoside, 3-O-glycoside, or 3,5-O-diglycoside, respectively; each sugar is selected from arabinose, galactose, glucose, rhamnose, and rutinose; and other hydrogen atoms of the 2-phenylchromenylium cation are optionally and independently substituted with hydroxy or methoxy. In some specific embodiments, each flavylium of the one or more flavyliums is a substituted 2-phenylchromenylium selected from 5-desoxymalvidin, 5-desoxypeonidin, 6-hydroxycyanidin, antirrhinin, apigeninidin, aurantinidin, callistephin, capensinidin, chrysanthemin, columnidin, cyanidin, cyanin, delphin, delphinidin, delphinidin 3-O-rhamnoside, diosmetinidin, europinidin, fisetinidin, gesneridin, guibourtinidin, hirsutidin, ideain, kaempferidinidin, luteolinidin, malvidin, malvin, myrtillin, oenin, pelargonidin, pelargonin, peonidin, peonidin 3-O-glucoside, peonin, petunidin, petunidin 3-O-arabinoside, petunidin 3-O-galactoside, petunidin 3-O-glucoside, petunidin 3-O-rhamnoside, petunidin 3-O-rutinoside, petunin, primulin, pulchellidin, pulchellidin 3-O-glucoside, pulchellidin 3-rhamnoside, robinetinidin, rosinidin, tricetinidin, and tulipanin.

In some embodiments, the one or more flavyliums comprise one or more substituted 2-phenylchromenyliums, in which exactly 1 hydrogen atom of a 2-phenylchromenylium cation is substituted with glucose such that the flavylium is a 3-O-glucoside; and other hydrogen atoms of the 2-phenylchromenylium cation are optionally and independently substituted with hydroxy or methoxy. In some specific embodiments, the one or more flavyliums comprise one or more substituted 2-phenylchromenyliums selected from callistephin, chrysanthemin, myrtillin, oenin, peonidin 3-O-glucoside, petunidin 3-O-glucoside, and pulchellidin 3-O-glucoside.

In some embodiments, each flavylium of the one or more flavyliums is a substituted 2-phenylchromenylium, in which exactly 1 hydrogen atom of a 2-phenylchromenylium cation is substituted with glucose such that the flavylium is a 3-O-glucoside; and other hydrogen atoms of the 2-phenylchromenylium cation are optionally and independently substituted with hydroxy or methoxy. In some specific embodiments, each flavylium of the one or more flavyliums is a substituted 2-phenylchromenylium selected from callistephin, chrysanthemin, myrtillin, oenin, peonidin 3-O-glucoside, petunidin 3-O-glucoside, and pulchellidin 3-O-glucoside.

In some embodiments, the one or more flavyliums comprise one or more substituted 2-phenylchromenyliums, in which at least one hydrogen atom of a 2-phenylchromenylium cation is substituted with hydroxy or methoxy, and other hydrogen atoms of the 2-phenylchromenylium cation are optionally and independently substituted hydroxy or methoxy. In some specific embodiments, the one or more flavyliums comprise one or more substituted 2-phenylchromenyliums selected from 3-deoxyanthocyanidin, 5-desoxymalvidin, 5-desoxypeonidin, 6-hydroxycyanidin, apigeninidin, aurantinidin, capensinidin, columnidin, cyanidin, delphinidin, 15 diosmetinidin, europinidin, fisetinidin, gesneridin, guibourtinidin, hirsutidin, kaempferidinidin, luteolinidin, malvidin, pelargonidin, peonidin, petunidin, pulchellidin, robinetinidin, rosinidin, and tricetinidin.

In some embodiments, each flavylium of the one or more flavyliums is a substituted 2-phenylchromenylium, in which at least one hydrogen atom of a 2-phenylchromenylium cation is substituted hydroxy or methoxy, and other hydrogen atoms of the 2-phenylchromenylium cation are optionally and independently substituted with hydroxy or methoxy. In some specific embodiments, each flavylium of the one or more flavyliums is a substituted 2-phenylchromenylium selected from 3-deoxyanthocyanidin, 5-desoxymalvidin, 5-desoxypeonidin, 6-hydroxycyanidin, apigeninidin, aurantinidin, capensinidin, columnidin, cyanidin, delphinidin, diosmetinidin, europinidin, fisetinidin, gesneridin, guibourtinidin, hirsutidin, kaempferidinidin, luteolinidin, malvidin, pelargonidin, peonidin, petunidin, pulchellidin, robinetinidin, rosinidin, and tricetinidin.

In some embodiments, the one or more flavyliums comprise 5-desoxymalvidin.

In some embodiments, the one or more flavyliums comprise 5-desoxypeonidin.

In some embodiments, the one or more flavyliums comprise 6-hydroxycyanidin.

In some embodiments, the one or more flavyliums comprise antirrhinin.

In some embodiments, the one or more flavyliums comprise apigeninidin.

In some embodiments, the one or more flavyliums comprise aurantinidin.

In some embodiments, the one or more flavyliums comprise callistephin.

In some embodiments, the one or more flavyliums comprise capensinidin.

In some embodiments, the one or more flavyliums comprise chrysanthemin.

In some embodiments, the one or more flavyliums comprise columnidin.

In some embodiments, the one or more flavyliums comprise cyanidin.

In some embodiments, the one or more flavyliums comprise cyanidin 3-O-(6-acetyl)glucoside.

In some embodiments, the one or more flavyliums comprise cyanidin 3-O-(6-p-coumaroyl)glucoside.

In some embodiments, the one or more flavyliums comprise cyanin.

In some embodiments, the one or more flavyliums comprise delphin.

In some embodiments, the one or more flavyliums comprise delphinidin.

In some embodiments, the one or more flavyliums comprise delphinidin 3-O-(6-acetyl)glucoside.

In some embodiments, the one or more flavyliums comprise delphinidin 3-O-(6-p-coumaroyl)glucoside.

In some embodiments, the one or more flavyliums comprise delphinidin 3-O-rhamnoside.

In some embodiments, the one or more flavyliums comprise diosmetinidin.

In some embodiments, the one or more flavyliums comprise europinidin.

In some embodiments, the one or more flavyliums comprise fisetinidin.

In some embodiments, the one or more flavyliums comprise gesneridin.

In some embodiments, the one or more flavyliums comprise guibourtinidin.

In some embodiments, the one or more flavyliums comprise hirsutidin.

In some embodiments, the one or more flavyliums comprise ideain.

In some embodiments, the one or more flavyliums comprise kaempferidinidin.

In some embodiments, the one or more flavyliums comprise luteolinidin.

In some embodiments, the one or more flavyliums comprise malvidin.

In some embodiments, the one or more flavyliums comprise malvidin 3-O-(6-acetyl)glucoside.

In some embodiments, the one or more flavyliums comprise malvidin 3-O-(6-p-caffeoyl)glucoside.

In some embodiments, the one or more flavyliums comprise malvidin 3-O-(6-p-coumaroyl)glucoside.

In some embodiments, the one or more flavyliums comprise malvin.

In some embodiments, the one or more flavyliums comprise myrtillin.

In some embodiments, the one or more flavyliums comprise oenin.

In some embodiments, the one or more flavyliums comprise pelargonidin.

In some embodiments, the one or more flavyliums comprise pelargonin.

In some embodiments, the one or more flavyliums comprise peonidin.

In some embodiments, the one or more flavyliums comprise peonidin 3-O-(6-acetyl)glucoside.

In some embodiments, the one or more flavyliums comprise peonidin 3-O-(6-p-caffeoyl)glucoside.

In some embodiments, the one or more flavyliums comprise peonidin 3-O-(6-p-coumaroyl)glucoside.

In some embodiments, the one or more flavyliums comprise peonidin 3-O-glucoside.

In some embodiments, the one or more flavyliums comprise peonin.

In some embodiments, the one or more flavyliums comprise petunidin.

In some embodiments, the one or more flavyliums comprise petunidin 3-O-(6-acetyl)galactoside.

In some embodiments, the one or more flavyliums comprise petunidin 3-O-(6-acetyl)glucoside.

In some embodiments, the one or more flavyliums comprise petunidin 3-O-(6-p-coumaroyl)glucoside.

In some embodiments, the one or more flavyliums comprise petunidin 3-O-arabinoside.

In some embodiments, the one or more flavyliums comprise petunidin 3-O-galactoside.

In some embodiments, the one or more flavyliums comprise petunidin 3-O-glucoside.

In some embodiments, the one or more flavyliums comprise petunidin 3-O-rhamnoside.

In some embodiments, the one or more flavyliums comprise petunidin 3-O-rutinoside.

In some embodiments, the one or more flavyliums comprise petunin.

In some embodiments, the one or more flavyliums comprise primulin.

In some embodiments, the one or more flavyliums comprise pulchellidin.

In some embodiments, the one or more flavyliums comprise pulchellidin 3-O-glucoside.

In some embodiments, the one or more flavyliums comprise pulchellidin 3-rhamnoside.

In some embodiments, the one or more flavyliums comprise robinetinidin.

In some embodiments, the one or more flavyliums comprise rosinidin.

In some embodiments, the one or more flavyliums comprise tricetinidin.

In some embodiments, the one or more flavyliums comprise tulipanin.

In some embodiments, the liquid phase comprises glycerol.

In some embodiments, the liquid phase comprises glycerol at a concentration of at least 5 percent by mass.

In some embodiments, the liquid phase comprises glycerol at a concentration of at least 10 percent by mass.

In some embodiments, the liquid phase comprises glycerol at a concentration of at least 20 percent by mass.

In some embodiments, the liquid phase comprises glycerol at a concentration of at least 30 percent by mass.

In some embodiments, the liquid phase comprises glycerol at a concentration of at least 40 percent by mass.

In some embodiments, the liquid phase comprises glycerol at a concentration of at least 50 percent by mass.

In some embodiments, the liquid phase comprises glycerol at a concentration of at least 5 percent and no greater than 88 percent by mass.

In some embodiments, the liquid phase comprises glycerol at a concentration of at least 5 percent and no greater than 80 percent by mass.

In some embodiments, the liquid phase comprises glycerol at a concentration of at least 20 percent and no greater than 93 percent by mass.

In some embodiments, the liquid phase comprises glycerol at a concentration of at least 30 percent and no greater than 60 percent by mass.

In some embodiments, the liquid phase comprises ethanol.

In some embodiments, the liquid phase comprises ethanol at a concentration of at least 1 percent by mass.

In some embodiments, the liquid phase comprises ethanol at a concentration of at least 5 percent by mass.

In some embodiments, the liquid phase comprises ethanol at a concentration of at least 10 percent by mass.

In some embodiments, the liquid phase comprises ethanol at a concentration of no greater than 50 percent by mass.

In some embodiments, the liquid phase comprises ethanol at a concentration of at least 1 percent and no greater than 40 percent by mass.

In some embodiments, the liquid phase comprises ethanol at a concentration of at least 5 percent and no greater than 50 percent by mass.

In some embodiments, the liquid phase comprises ethanol at a concentration of at least 5 percent and no greater than 40 percent by mass.

In some embodiments, the liquid phase comprises water.

In some embodiments, the liquid phase comprises water at a concentration of at least 5 parts per million by mass.

In some embodiments, the liquid phase comprises water at a concentration of at least 0.5 percent by mass.

In some embodiments, the liquid phase comprises water at a concentration of at least 2 percent by mass.

In some embodiments, the liquid phase comprises water at a concentration of no greater than 25 percent by mass.

In some embodiments, the liquid phase comprises water at a concentration of no greater than 20 percent by mass.

In some embodiments, the liquid phase comprises water at a concentration of at least 5 parts per million and no greater than 20 percent by mass.

In some embodiments, the liquid phase comprises water at a concentration of at least 0.5 percent and no greater than 20 percent by mass.

In some embodiments, the liquid phase comprises water at a concentration of at least 2 percent and no greater than 25 percent by mass.

In some embodiments, the liquid phase comprises water at a concentration of at least 2 percent and no greater than 20 percent by mass.

In some embodiments, the liquid phase comprises propylene glycol.

In some embodiments, the liquid phase comprises propylene glycol at a concentration of at least 1 percent by mass.

In some embodiments, the liquid phase comprises propylene glycol at a concentration of no greater than 25 percent by mass.

In some embodiments, the liquid phase comprises propylene glycol at a concentration of no greater than 20 percent by mass.

In some embodiments, the liquid phase comprises propylene glycol at a concentration of at least 1 percent and no greater than 25 percent by mass.

In some embodiments, the liquid phase comprises propylene glycol at a concentration of at least 1 percent and no greater than 20 percent by mass.

In some embodiments, the liquid phase comprises the one or more flavyliums at a combined concentration of at least 0.1 percent by mass.

In some embodiments, the liquid phase comprises the one or more flavyliums at a combined concentration of no greater than 30 percent by mass.

In some embodiments, the liquid phase comprises the one or more flavyliums at a combined concentration of at least 0.1 percent and no greater than 15 percent by mass.

In some embodiments, the liquid phase comprises the tetrahydrocannabinol at a concentration of at least 0.1 percent and no greater than 7.5 percent by mass.

In some embodiments, the liquid phase comprises the tetrahydrocannabinol at a concentration of at least 0.1 percent and no greater than 3.5 percent by mass.

In some embodiments, the composition lacks lipids at a concentration greater than 5 percent by mass.

In some embodiments, the composition lacks a lipid phase at a concentration greater than 5 percent by mass.

In some embodiments, the composition lacks a lipid phase at a concentration greater than 1 percent by mass.

In some embodiments, the composition lacks a lipid phase at a concentration greater than 0.2 percent by mass.

In some embodiments, the composition lacks a lipid phase.

In some embodiments, the composition lacks triglycerides at a concentration greater than 5 percent by mass.

In some embodiments, the composition lacks triglycerides at a concentration greater than 1 percent by mass.

In some embodiments, the composition lacks triglycerides at a concentration greater than 0.2 percent by mass.

In some embodiments, the composition lacks triglycerides.

In some embodiments, the composition lacks surfactants that are not flavyliums at a concentration greater than 5 percent by mass.

In some embodiments, the composition lacks surfactants that are not flavyliums at a concentration greater than 1 percent by mass.

In some embodiments, the composition lacks surfactants that are not flavyliums at a concentration greater than 0.2 percent by mass.

In some embodiments, the composition lacks surfactants that are not flavyliums.

In some embodiments, the liquid phase lacks chemical species that have a molecular weight of greater than 2000 atomic mass units at a concentration greater than 5 percent by mass.

In some embodiments, the liquid phase lacks chemical species that have a molecular weight of greater than 2000 atomic mass units at a concentration greater than 1 percent by mass.

In some embodiments, the liquid phase lacks chemical species that have a molecular weight of greater than 2000 atomic mass units at a concentration greater than 0.2 percent by mass.

In some embodiments, the composition lacks chemical species that have a molecular weight of greater than 2000 atomic mass units.

In some embodiments, tetrahydrocannabinol has a solubility in the liquid phase; tetrahydrocannabinol has a solubility in a reference liquid phase that lacks the one or more flavyliums and that is otherwise identical to the liquid phase; and the solubility of tetrahydrocannabinol in the liquid phase is greater than the solubility of tetrahydrocannabinol in the reference liquid phase.

In some embodiments, tetrahydrocannabinol has a solubility in a reference liquid phase that lacks the one or more flavyliums and that is otherwise identical to the liquid phase; and the concentration of the tetrahydrocannabinol in the liquid phase is greater than the solubility of tetrahydrocannabinol in the reference liquid phase.

In some embodiments, the tetrahydrocannabinol and the one or more flavyliums undergo attractive, noncovalent interactions in the liquid phase.

In some embodiments, the tetrahydrocannabinol and the one or more flavyliums undergo pi-stacking interactions in the liquid phase.

In some embodiments, the tetrahydrocannabinol and the one or more flavyliums undergo hydrogen bonding interactions in the liquid phase.

In some embodiments, the tetrahydrocannabinol lacks a net charge; and each flavylium of the one or more flavyliums has an electrical charge of +1.

In some embodiments, each flavylium of the one or more flavyliums has an electrical charge of +1.

In some embodiments, the tetrahydrocannabinol is not present in a salt form in the liquid phase.

In some embodiments, no flavylium of the one or more flavyliums is present in a salt form in the liquid phase.

In some embodiments, the composition comprises either cherries or an extract thereof, wherein at least one flavylium of the one or more flavyliums is derived from the cherries or the extract thereof.

In some embodiments, the composition comprises either tart cherries or an extract thereof, wherein at least one flavylium of the one or more flavyliums is derived from the tart cherries or the extract thereof.

In some embodiments, the composition comprises cherry juice, wherein at least one flavylium of the one or more flavyliums is derived from the cherry juice.

In some embodiments, the composition comprises tart cherry juice, wherein at least one flavylium of the one or more flavyliums is derived from the tart cherry juice.

In some embodiments, the composition comprises cherry powder, wherein at least one flavylium of the one or more flavyliums is derived from the cherry powder.

In some embodiments, the composition comprises tart cherry powder, wherein at least one flavylium of the one or more flavyliums is derived from the tart cherry powder.

In some embodiments, the composition comprises either acai berries or an extract thereof, wherein at least one flavylium of the one or more flavyliums is derived from the acai berries or the extract thereof.

In some embodiments, the composition comprises either blackberries or an extract thereof, wherein at least one flavylium of the one or more flavyliums is derived from the blackberries or the extract thereof.

In some embodiments, the composition comprises either raspberries or an extract thereof, wherein at least one flavylium of the one or more flavyliums is derived from the raspberries or the extract thereof.

In some embodiments, the composition comprises either black rice or an extract thereof, wherein at least one flavylium of the one or more flavyliums is derived from the black rice or the extract thereof.

In some embodiments, the composition comprises either soybeans or an extract thereof, wherein at least one flavylium of the one or more flavyliums is derived from the soybeans or the extract thereof.

In some embodiments, the composition comprises either blueberries or an extract thereof, wherein at least one flavylium of the one or more flavyliums is derived from the blueberries or the extract thereof.

In some embodiments, the composition comprises either blue corn or an extract thereof, wherein at least one flavylium of the one or more flavyliums is derived from the blue corn or the extract thereof.

In some embodiments, the composition comprises either grapes or an extract thereof, wherein at least one flavylium of the one or more flavyliums is derived from the grapes or the extract thereof. In some embodiments, the composition comprises either cranberry or an extract thereof, wherein at least one flavylium of the one or more flavyliums is derived from the cranberry or the extract thereof.

In some embodiments, the composition comprises either eggplants or an extract thereof, wherein at least one flavylium of the one or more flavyliums is derived from the eggplants or the extract thereof.

In some embodiments, the composition comprises either plums or an extract thereof, wherein at least one flavylium of the one or more flavyliums is derived from the plums or the extract thereof.

In some embodiments, the composition comprises either pomegranate or an extract thereof, wherein at least one flavylium of the one or more flavyliums is derived from the pomegranate or the extract thereof.

In some embodiments, the composition comprises either red cabbage or an extract thereof, wherein at least one flavylium of the one or more flavyliums is derived from the red cabbage or the extract thereof.

In some embodiments, the composition comprises either red currants or an extract thereof, wherein at least one flavylium of the one or more flavyliums is derived from the red currants or the extract thereof.

In some embodiments, the composition comprises either red onions or an extract thereof, wherein at least one flavylium of the one or more flavyliums is derived from the red onions or the extract thereof.

In some embodiments, the composition comprises either tomatoes or an extract thereof, wherein at least one flavylium of the one or more flavyliums is derived from the tomatoes or the extract thereof.

In some embodiments, each flavylium of the one or more flavyliums is derived from cherries.

In some embodiments, each flavylium of the one or more flavyliums is derived from tart cherries.

In some embodiments, each flavylium of the one or more flavyliums is derived from cherry powder.

In some embodiments, each flavylium of the one or more flavyliums is derived from tart cherry powder.

In some embodiments, each flavylium of the one or more flavyliums is derived from cherry juice.

In some embodiments, each flavylium of the one or more flavyliums is derived from tart cherry juice.

In some embodiments, each flavylium of the one or more flavyliums has a molecular weight of at least 239 and no greater than 900 atomic mass units.

In some embodiments, each flavylium of the one or more flavyliums has a molecular weight of at least 239 and no greater than 750 atomic mass units.

In some embodiments, each flavylium of the one or more flavyliums has a concentration in the liquid phase; the one or more flavyliums have a combined concentration in the liquid phase that is equal to the sum of the concentrations of each flavylium in the liquid phase; and the liquid phase has a ratio by mass of the combined concentration of the one or more flavyliums to the concentration of the tetrahydrocannabinol that is greater than 1:100 (flavyliums:tetrahydrocannabinol).

In some embodiments, each flavylium of the one or more flavyliums has a concentration in the liquid phase; the one or more flavyliums have a combined concentration in the liquid phase that is equal to the sum of the concentrations of each flavylium in the liquid phase; and the liquid phase has a ratio by mass of the combined concentration of the one or more flavyliums to the concentration of the tetrahydrocannabinol that is greater than 1:10 (flavyliums:tetrahydrocannabinol).

In some embodiments, each flavylium of the one or more flavyliums has a concentration in the liquid phase; the one or more flavyliums have a combined concentration in the liquid phase that is equal to the sum of the concentrations of each flavylium in the liquid phase; and the liquid phase has a ratio by mass of the combined concentration of the one or more flavyliums to the concentration of the tetrahydrocannabinol that is greater than 1:1 (flavyliums:tetrahydrocannabinol).

In some embodiments, each flavylium of the one or more flavyliums has a concentration in the liquid phase; the one or more flavyliums have a combined concentration in the liquid phase that is equal to the sum of the concentrations of each flavylium in the liquid phase; and the liquid phase has a ratio by mass of the combined concentration of the one or more flavyliums to the concentration of the tetrahydrocannabinol that is at least 3:2 (flavyliums:tetrahydrocannabinol).

In some embodiments, each flavylium of the one or more flavyliums has a concentration in the liquid phase; the one or more flavyliums have a combined concentration in the liquid phase that is equal to the sum of the concentrations of each flavylium in the liquid phase; and the liquid phase has a ratio by mass of the combined concentration of the one or more flavyliums to the concentration of the tetrahydrocannabinol that is at least 1:100 and no greater than 3:1 (flavyliums:tetrahydrocannabinol).

In some embodiments, the composition is formulated for human consumption; the composition is formulated for oral administration; and liquid phase comprises at least 3 kilocalories of nutritional energy per gram.

In some embodiments, the composition is formulated for human consumption; the composition is formulated for oral administration; and the liquid phase comprises no greater than 6 kilocalories of nutritional energy per gram.

In some embodiments, the composition is formulated for human consumption; the composition is formulated for oral administration; and the liquid phase comprises at least 4 kilocalories and no greater than 5 kilocalories of nutritional energy per gram.

In some embodiments, the liquid phase comprises a concentration of ethyl oxonium and a concentration of ethoxide at a ratio of at least 1:100 by mole.

In some embodiments, the liquid phase comprises a concentration of ethyl oxonium and a concentration of ethoxide at a ratio of at least 10,000:1 and no greater than 1,000,000:1 by mole.

In some embodiments, the liquid phase comprises a concentration of ethyl oxonium and a concentration of ethoxide at a ratio of at least 1,000:1 and no greater than 100,000:1 by mole.

In some embodiments, the liquid phase comprises a concentration of ethyl oxonium and a concentration of ethoxide at a ratio of at least 100:1 and no greater than 10,000:1 by mole.

In some embodiments, the liquid phase comprises a concentration of ethyl oxonium and a concentration of ethoxide at a ratio of at least 10:1 and no greater than 1,000:1 by mole.

In some embodiments, the liquid phase comprises a concentration of ethyl oxonium and a concentration of ethoxide at a ratio of at least 1:1 and no greater than 100:1 by mole.

In some embodiments, the liquid phase comprises a concentration of ethyl oxonium and a concentration of ethoxide at a ratio of at least 1:10 and no greater than 10:1 by mole.

In some embodiments, the liquid phase comprises a concentration of ethyl oxonium and a concentration of ethoxide at a ratio of at least 1:100 and no greater than 1:1 by mole.

In some embodiments, the liquid phase comprises a concentration of hydronium and a concentration of hydroxide at a ratio of at least 1:100 by mole.

In some embodiments, the liquid phase comprises a concentration of hydronium and a concentration of hydroxide at a ratio of at least 10,000:1 and no greater than 1,000,000:1 by mole.

In some embodiments, the liquid phase comprises a concentration of hydronium and a concentration of hydroxide at a ratio of at least 1,000:1 and no greater than 100,000:1 by mole.

In some embodiments, the liquid phase comprises a concentration of hydronium and a concentration of hydroxide at a ratio of at least 100:1 and no greater than 10,000:1 by mole.

In some embodiments, the liquid phase comprises a concentration of hydronium and a concentration of hydroxide at a ratio of at least 10:1 and no greater than 1,000:1 by mole.

In some embodiments, the liquid phase comprises a concentration of hydronium and a concentration of hydroxide at a ratio of at least 1:1 and no greater than 100:1 by mole.

In some embodiments, the liquid phase comprises a concentration of hydronium and a concentration of hydroxide at a ratio of at least 1:10 and no greater than 10:1 by mole.

In some embodiments, the liquid phase comprises a concentration of hydronium and a concentration of hydroxide at a ratio of at least 1:100 and no greater than 1:1 by mole.

In some embodiments, the composition is formulated for human consumption; the composition is formulated for oral administration; and the composition if formulated such that a portion of the tetrahydrocannabinol of the composition is absorbed into the epithelial lining of the gastrointestinal tract of a human following oral administration by the human before the composition enters the stomach of the human.

In some embodiments, the composition is formulated for human consumption; the composition is formulated for oral administration; and the composition if formulated such that at least 10 percent of the tetrahydrocannabinol of the composition is absorbed into the epithelial lining of the gastrointestinal tract of a human following oral administration by the human before the composition enters the stomach of the human.

In some embodiments, the composition is formulated for human consumption; the composition is formulated for oral administration; and the composition if formulated such that at least 20 percent of the tetrahydrocannabinol of the composition is absorbed into the epithelial lining of the gastrointestinal tract of a human following oral administration by the human before the composition enters the stomach of the human.

In some embodiments, the composition is formulated for human consumption; the composition is formulated for oral administration; and the composition if formulated such that at least 30 percent of the tetrahydrocannabinol of the composition is absorbed into the epithelial lining of the gastrointestinal tract of a human following oral administration by the human before the composition enters the stomach of the human.

In some embodiments, the composition is formulated for human consumption; the composition is formulated for oral administration; the composition comprises an oral bioavailability of at least 10 percent; and the oral bioavailability is a percentage of the tetrahydrocannabinol of the composition that would be expected to enter the blood of a human following oral administration by the human to the human.

In some embodiments, the composition is formulated for human consumption; the composition is formulated for oral administration; the composition comprises an oral bioavailability of at least 20 percent; and the oral bioavailability is a percentage of the tetrahydrocannabinol of the composition that would be expected to enter the blood of a human following oral administration by the human to the human.

In some embodiments, the composition is formulated for human consumption; the composition is formulated for oral administration; the composition comprises an oral bioavailability of at least 30 percent; and the oral bioavailability is a percentage of the tetrahydrocannabinol of the composition that would be expected to enter the blood of a human following oral administration by the human to the human.

In some embodiments, the composition is formulated for human consumption; the composition is formulated for oral administration; the composition comprises a Tmax that is no greater than 30 minutes; and the Tmax is the time to achieve a maximum blood concentration of tetrahydrocannabinol in a human following oral administration.

In some embodiments, the composition is formulated for human consumption; the composition is formulated for oral administration; the composition comprises a Tmax that is no greater than 20 minutes; and the Tmax is the time to achieve a maximum blood concentration of tetrahydrocannabinol in a human following oral administration.

In some embodiments, the composition is formulated for human consumption; the composition is formulated for oral administration; the composition comprises a Tmax that is no greater than 10 minutes; and the Tmax is the time to achieve a maximum blood concentration of tetrahydrocannabinol in a human following oral administration.

In some embodiments, the oral administration is sublingual, sublabial, or buccal administration.

In some embodiments, the composition is formulated such that at least a portion of the tetrahydrocannabinol of the composition is absorbed by the epithelial lining of the gastrointestinal tract of a human upstream of the stomach.

In some embodiments, the composition is formulated such that a majority of the tetrahydrocannabinol of the composition is absorbed by the epithelial lining of the gastrointestinal tract of a human upstream of the stomach.

In some embodiments, the composition is formulated such that a portion of the tetrahydrocannabinol of the composition is absorbed by the epithelial lining of one or both of the mouth and esophagus of a human.

In some embodiments, the composition is formulated such that a majority of the tetrahydrocannabinol of the composition is absorbed by the epithelial lining of one or both of the mouth and esophagus of a human.

In some embodiments, the composition is for use as a medicament.

In some embodiments, the composition is for use to manufacture a medicament.

In some embodiments, the composition is a spray.

In some embodiments, the composition is a spray; and the spray comprises at least 20 micrograms and no greater than 6 milligrams of the tetrahydrocannabinol.

In some embodiments, the composition is a spray; and the spray comprises at least 800 micrograms and no greater than 1.2 milligrams of the tetrahydrocannabinol.

In some embodiments, the composition is a spray; and the spray comprises at least 20 microliters and no greater than 250 microliters of the liquid phase.

In some embodiments, the composition is a spray; and the spray comprises at least 80 microliters and no greater than 190 microliters of the liquid phase.

In some embodiments, the composition is an aerosol spray.

Various aspects of this disclosure relate to a container that contains a composition as described anywhere in the disclosure, wherein the container is configured to produce the spray as described in any of the preceding six paragraphs.

Various aspects of this disclosure relate to a container that contains a composition as described anywhere in the disclosure.

In some embodiments, the container contains at least 100 microliters and no greater than 50 milliliters of the composition.

In some embodiments, the container contains at least 9 milliliters and no greater than 25 milliliters of the composition.

In some embodiments, the container contains no greater than 16 milliliters of the composition.

In some embodiments, the composition contained by the container comprises at least 9 milligrams of the tetrahydrocannabinol.

In some embodiments, the composition contained by the container comprises no greater than 120 milligrams of the tetrahydrocannabinol.

In some embodiments, the composition contained by the container comprises at least 84 milligrams and no greater than 116 milligrams of the tetrahydrocannabinol.

In some embodiments, the composition contained by the container comprises no greater than 110 milligrams of the tetrahydrocannabinol.

In some embodiments, air surrounds the container; and the composition is hermetically sealed within the container such that the composition lacks fluid communication with the air that surrounds the container.

In some embodiments, the container is configured to spray an amount of the composition from the container.

In some embodiments, the container is configured to spray the composition from the container in an amount of at least 20 microliters and no greater than 250 microliters per spray.

In some embodiments, the container is configured to spray the composition from the container in an amount of at least 80 microliters and no greater than 190 microliters per spray.

Various aspects of this disclosure relate to a method to administer tetrahydrocannabinol to a human, comprising providing a container as described anywhere in this disclosure; and orally administering an amount of the composition from the container to the human.

Various aspects of this disclosure relate to a method to administer tetrahydrocannabinol to a human, comprising providing a composition as described anywhere in this disclosure; and orally administering an amount of the composition to the human.

In some embodiments, orally administering an amount of the composition comprises spraying the amount of the composition from a container into the mouth of the human.

In some embodiments, the amount is at least 20 microliters and no greater than 250 microliters of the composition.

In some embodiments, the amount is at least 80 microliters and no greater than 190 microliters of the composition.

In some embodiments, the amount comprises at least 20 micrograms and no greater than 6 milligrams of the tetrahydrocannabinol.

In some embodiments, the amount comprises at least 800 micrograms and no greater than 1.2 milligrams of the tetrahydrocannabinol.

In some embodiments, the oral administration is sublingual, sublabial, or buccal administration.

### EXEMPLIFICATION

The following example illustrates a commercially-relevant embodiment, and the example does not limit the disclosure or any patent claim that matures from this disclosure.

The Example. The manufacture of an oral tetrahydrocannabinol spray comprising flavylium excipients.

3.4 grams of marijuana distillate and 18 grams of tart cherry powder (BulkSupplements.com, Nevada, United States) were dissolved in 116 grams of 190 proof ethanol and 47 grams of propylene glycol. The marijuana distillate contained 82 percent tetrahydrocannabinol, and the tart cherry powder contained about 2 percent flavyliums derived Prunus cerasus fruit. 263 grams of glycerol was then added to the solution, and the solution was mixed for 10 minutes.

The solution described in the preceding paragraph was loaded into 15 milliliter spray bottles for oral administration. Each spray bottle produced a spray containing approximately 150 microliters of a liquid phase in which each 150 microliter spray contained approximately 1 milligram of tetrahydrocannabinol.

What is claimed is:

1. A composition, comprising a liquid phase that consists of chemical species that comprise tetrahydrocannabinol, one or more flavyliums, and a solvent, wherein each chemical species of the liquid phase has a concentration in the liquid phase; the tetrahydrocannabinol is dissolved in the liquid phase at a concentration of at least 0.1 percent and no greater than 8 percent by mass; each flavylium of the one or more flavyliums is a substituted 2-phenylchromenylium that consists of carbon, hydrogen, and oxygen atoms; the solvent is miscible with water; and the liquid phase comprises the solvent at a greater concentration by mass than any other chemical species of the liquid phase.

2. The composition of claim 1, wherein each flavylium of the one or more flavyliums is a substituted 2-phenylchromenylium, in which 0, 1, or 2 hydrogen atoms of a 2-phenylchromenylium cation are independently substituted with a sugar such that the flavylium is an aglycoside, 3-O-glycoside, or 3,5-O-diglycoside, respectively; each sugar is selected from 6-O-acetylglucose, 6-O-(para-caffeoyl)glucose, 6-O-(para-coumaroyl)glucose, arabinose, galactose, glucose, rhamnose, and rutinose; and other hydrogen atoms of the 2-phenylchromenylium cation are optionally and independently substituted with hydroxy or methoxy.

3. The composition of claim 1, wherein each flavylium of the one or more flavyliums is a substituted 2-phenylchromenylium selected from 5-desoxymalvidin, 5-desoxypeonidin, 6-hydroxycyanidin, antirrhinin, apigeninidin, aurantinidin, callistephin, capensinidin, chrysanthemin, columnidin, cyanidin, cyanidin 3-O-(6-acetyl)glucoside, cyanidin 3-O-(6-p-coumaroyl)glucoside, cyanin, delphin, delphinidin, delphinidin 3-O-(6-acetyl)glucoside, delphinidin 3-O-(6-p-coumaroyl)glucoside, delphinidin 3-O-rhamnoside, diosmetinidin, europinidin, fisetinidin, gesneridin, guibourtinidin, hirsutidin, ideain, kaempferidinidin, luteolinidin, malvidin, malvidin 3-O-(6-acetyl)glucoside, malvidin 3-O-(6-p-caffeoyl)glucoside, malvidin 3-O-(6-p-coumaroyl)glucoside, malvin, myrtillin, oenin, pelargonidin, pelargonin, peonidin, peonidin acetyl)glucoside, peonidin 3-O-(6-p-caffeoyl)glucoside, peonidin 3-O-(6-p-coumaroyl)glucoside, peonidin 3-O-glucoside, peonin, petunidin, petunidin 3-O-(6-acetyl)galactoside, petunidin 3-O-(6-acetyl)glucoside, petunidin 3-O-(6-p-coumaroyl)glucoside, petunidin 3-O-arabinoside, petunidin 3-O-galactoside, petunidin 3-O-glucoside, petunidin 3-O-rhamnoside, petunidin 3-O-rutinoside, petunin, primulin, pulchellidin, pulchellidin 3-O-glucoside, pulchellidin 3-rhamnoside, robinetinidin, rosinidin, tricetinidin, and tulipanin.

4. The composition of claim 1, wherein the one or more flavyliums comprise one or more substituted 2-phenylchromenyliums, in which 0, 1, or 2 hydrogen atoms of a 2-phenylchromenylium cation are independently substituted with a sugar such that the flavylium is an aglycoside, 3-O-glycoside, or 3,5-O-diglycoside, respectively; each sugar is selected from arabinose, galactose, glucose, rhamnose, and rutinose; and other hydrogen atoms of the 2-phenylchromenylium cation are optionally and independently substituted with hydroxy or methoxy.

5. The composition of claim 1, wherein each flavylium of the one or more flavyliums is a substituted 2-phenylchromenylium selected from 5-desoxymalvidin, 5-desoxypeonidin, 6-hydroxycyanidin, antirrhinin, apigeninidin, aurantinidin, callistephin, capensinidin, chrysanthemin, columnidin, cyanidin, cyanin, delphin, delphinidin, delphinidin 3-O-rhamnoside, diosmetinidin, europinidin, fisetinidin, gesneridin, guibourtinidin, hirsutidin, ideain, kaempferidinidin, luteolinidin, malvidin, malvin, myrtillin, oenin, pelargonidin, pelargonin, peonidin, peonidin 3-O-glucoside, peonin, petunidin, petunidin 3-O-arabinoside, petunidin 3-O-galactoside, petunidin 3-O-glucoside, petunidin 3-O-rhamnoside, petunidin 3-O-rutinoside, petunin, primulin, pulchellidin, pulchellidin 3-O-glucoside, pulchellidin 3-rhamnoside, robinetinidin, rosinidin, tricetinidin, and tulipanin.

6. The composition of claim 1, wherein each flavylium of the one or more flavyliums is a substituted 2-phenylchromenylium selected from callistephin, chrysanthemin, myrtillin, oenin, peonidin 3-O-glucoside, petunidin 3-O-glucoside, and pulchellidin 3-O-glucoside.

7. The composition of claim 1, wherein each flavylium of the one or more flavyliums is a substituted 2-phenylchromenylium selected from 3-deoxyanthocyanidin, 5-desoxymalvidin, 5-desoxypeonidin, 6-hydroxycyanidin, apigeninidin, aurantinidin, capensinidin, columnidin, cyanidin, delphinidin, diosmetinidin, europinidin, fisetinidin, gesneridin, guibourtinidin, hirsutidin, kaempferidinidin, luteolinidin, malvidin, pelargonidin, peonidin, petunidin, pulchellidin, robinetinidin, rosinidin, and tricetinidin.

8. The composition of claim 1, wherein the liquid phase comprises glycerol at a concentration of at least 20 percent and no greater than 93 percent by mass.

9. The composition a of claim 1, wherein the liquid phase comprises ethanol at a concentration of at least 5 percent and no greater than 40 percent by mass.

10. The composition of claim 1, wherein the liquid phase comprises water at a concentration of at least 0.5 percent and no greater than 20 percent by mass.

11. The composition of claim 1, comprising tart cherry powder, wherein at least one flavylium of the one or more flavyliums is derived from the tart cherry powder.

12. The composition of claim 1, wherein each flavylium of the one or more flavyliums has a concentration in the liquid phase; the one or more flavyliums have a combined concentration in the liquid phase that is equal to the sum of the concentrations of each flavylium in the liquid phase; and the liquid phase has a ratio by mass of the combined concentration of the one or more flavyliums to the concentration of the tetrahydrocannabinol that is greater than 1:1 (flavyliums:tetrahydrocannabinol).

13. The composition of claim 1, wherein the composition is formulated for human consumption; the composition is formulated for oral administration; and the liquid phase comprises at least 4 kilocalories and no greater than 5 kilocalories of nutritional energy per gram.

14. The composition of claim 1, wherein the liquid phase comprises a concentration of ethyl oxonium and a concentration of ethoxide at a ratio of at least 100:1 and no greater than 10,000:1 by mole.

15. The composition of claim 1, wherein the composition is formulated for human consumption; the composition is formulated for oral administration; and the composition if formulated such that a portion of the tetrahydrocannabinol of the composition is absorbed into the epithelial lining of the gastrointestinal tract of a human following oral administration by the human before the composition enters the stomach of the human.

16. The composition of claim 1, wherein the composition is formulated for human consumption; the composition is formulated for oral administration; the composition comprises an oral bioavailability of at least 10 percent; and the oral bioavailability is a percentage of the tetrahydrocannabinol of the composition that would be expected to enter the blood of a human following oral administration by the human to the human.

17. The composition of claim 1, wherein the composition is formulated for human consumption; the composition is formulated for oral administration; the composition comprises a Tmax that is no greater than 30 minutes; and the Tmax is the time to achieve a maximum blood concentration of tetrahydrocannabinol in a human following oral administration.

18. The composition of claim 1, wherein the composition is a spray; and the spray comprises at least 800 micrograms and no greater than 1.2 milligrams of the tetrahydrocannabinol.

19. The composition of claim 1, wherein the composition is a spray; and the spray comprises at least 80 microliters and no greater than 190 microliters of the liquid phase.

20. The composition of claim 1, wherein the composition is an aerosol spray.
